(19) United States
(12) Patent Application Publication
(10) Pub. No.: US 2022/0331391 A1
(43) Pub. Date: Oct. 20, 2022

(54) METHODS AND COMPOSITIONS FOR TREATING CENTRAL NERVOUS SYSTEM COCCIDIOIDOMYCOSIS WITH NIKKOMYCIN Z MICRO DOSING

(71) Applicant: David J. Larwood, Woodside, CA (US)

(72) Inventor: David J. Larwood, Woodside, CA (US)

(21) Appl. No.: 17/722,241

(22) Filed: Apr. 15, 2022

Related U.S. Application Data
(60) Provisional application No. 63/175,594, filed on Apr. 16, 2021.

(51) Int. Cl. A61K 38/05; A61P 31/10; A61K 38/00; A61K 9/00

(52) U.S. Cl. CPC A61K 38/05; A61P 31/10; A61K 38/005; A61K 9/0095; A61K 9/0053

(57) ABSTRACT

Nikkomycin Z (nikZ) is an antifungal agent that clears from a mammalian bloodstream relatively rapidly, with a clearance half-life of about 2 to 2.5 hours in humans. Supplying additional drug at less than two clearance half-lives (about 4 hours) and better still at less than one half-life (about 2 hours) can replace cleared drug to better maintain a steady blood level. Such a schedule is challenging in humans and not practical in treating mice. Providing a continuous supply of drug in water (“DiW”), nikZ dissolved in drinking water permits rodents to drink ad libitum, proved highly efficacious in treating intracranial infection with coccidioidomycosis. Comparison with a similar study dosing nikZ TID, the DiW dosing showed the same reduction of fungal burden at a >95% lower dose and similar survival at a >75% reduction of dose compared with TID dosing.

### FIELD OF THE INVENTION

This invention relates to a method of administering drug in a finely divided dosing schedule and mechanism to provide more consistent blood levels and more effective utilization of a provided drug compared to repeat dosing at intervals ≥6 hours. This invention enables both a more consistent level of drug in the blood and a convenient method to ascertain a just-sufficient effective level of drug for a therapeutic regime.

The present invention relates generally to a method of using compounds capable of inhibiting chitin synthase to treat fungal infections in mammals. In one preferred embodiment, the present invention is directed to the use of a class of compounds known as nikkomycins to treat infections of Coccidioides spp. in mammals, particularly in the central nervous system.

### CROSS REFERENCE TO RELATED APPLICATIONS

This application is based on and claims benefit from provisional application “Methods And Compositions For Treating Central Nervous System Coccidioidomycosis With Nikkomycin Z Micro Dosing” filed Apr. 16, 2021 as U.S. Provisional Application No. 63/175,594. (the “'594 application”).

This application is based in part on and draws from the principals and methods disclosed in “Methods and Compositions for Microdosing, Simulating Extended-Release Formulations”, filed Aug. 5, 2020, as U.S. Provisional Application No. 63/061,789 (the “789 application”) and filed Aug. 5, 2021 as U.S. application Ser. No. 17/395,254 (the “'254 application”). This application is based in part on and draws from the principals and methods disclosed in “Methods and Compositions for Treating disseminated coccidioidomycosis with Nikkomycin Z Micro dosing”, filed Apr. 13, 2021, as U.S. Provisional Application No. 63/174,537 (the “537 application”) and filed Apr. 13, 2022 as U.S. application Ser. No. 17/719,425 (the “'425 application”). The present application incorporates the '594, '789, '254, '537 and '425 applications by reference and claims priority therefrom.

### BACKGROUND OF THE INVENTION

Coccidioidal meningitis is the most feared and most lethal complication of disseminated coccidioidomycosis [1]. The many complications include hydrocephalus, vasculitis, cerebral or spinal cord infarction, arachnoiditis, cranial nerve palsy, syringomyelia, transverse myelitis, cord compression, paralyses, parenchymal abscesses, and seizures [2]. Oral azole therapy is the most common treatment modality for coccidioidal meningitis [2], but such treatment requires lifetime administration to suppress recurrences [3]. In many cases intrathecal amphotericin is required to stop progression, but this form of therapy is inconvenient and neurotoxic [4]. Incidence is reported to be 200-500 new human cases per year [30].

Nikkomycin Z (nikZ) is a water-soluble nucleoside peptide, originally derived from Streptomyces tendae [5]. NikZ enters the fungal cell via peptide permeases [5]. NikZ is structurally similar to UDP-N-acetylglucosamine, enabling it to be a competitive inhibitor for the enzyme chitin synthase, an enzyme that processes UDP-N-acetylglucosamine to chitin, an important structural component in fungal cell walls [5]. NikZ is particularly inhibitory to the dimorphic fungi [6-10].

NikZ appears to have a very low order of toxicity; as studied in mice, oral doses up to 2000 mg/kg/day have been tolerated [8, 11, 11b]. Against coccidioidomycosis, striking oral nikZ antifungal efficacy has been demonstrated in murine models of pulmonary, central nervous system or disseminated disease [6, 7, 11, 11b, 12].

A concern with possible future therapy with nikZ in humans is its half-life in blood clearance of approximately 1-2 hours, in both mice and humans [13-15]. This has resulted in the use of multiple dose/day regimens in animal experiments, shown superior to once/day administration [6]. The use of multiple dose/day regimens has thus also been projected for human use [6]. This study of efficacy of nikZ against the most severe form of coccidioidal disease, central nervous system infection examined the possibility that an extended-release form of the drug, one that would provide steady state blood levels for a therapy duration, while relinquishing bolus pulses of drug, could prove efficacious. Extended release would enhance convenience for and adherence to administration to patients, compared to multiple dose/day regimens, and possibly also improve efficacy. This simulated a possible extended-release form by providing the drug ad libitum in drinking water to infected mice.

This application is the subject of a publication submitted Mar. 24, 2021 for review, published in September 2021 [32], as well as Mar. 21, 2021 as a brief abstract for a talk that was presented at the Coccidioidomycosis Study Group 65th annual meeting on Saturday, Apr. 17, 2021 [31].

### SUMMARY OF THE INVENTION

Meningitis is the most feared coccidioidomycosis complication. Nikkomycin Z (nikZ) is a chitin synthase inhibitor. A concern is short half-life, resulting in multiple dose/day experiment regimens. This studied an interim model of extended release, providing improved stability in blood levels, seeking the steady state blood levels anticipated with an extended-release formulation. Extended-release dosing QD or BID would enhance convenience, and adherence, for patients. This is simulated by providing nikZ continuously in drinking water.

Coccidioides was injected intracerebrally into mice. Treatment began day 3, for 12 days. Fluconazole was given 100 mg/kg QD by gavage; designed doses of nikZ 30, 100 or 300 mg/kg/day given in drinking water, and intake monitored. Post-treatment, mice were observed 30 days, then survivors euthanized, brain CFU quantitated, CFU in other organs assessed.

NikZ was stable in drinking water. Survival was 11%, 50%, 70%, 90% and 100% in untreated controls, fluconazole, and nikZ 30, 100 or 300 mg/kg/day groups, respectively. Brains were sterilized in 0%, 20%, 86%, 89%, 80% in survivors in these groups, or 0 of 1, 1 of 5, 6 of 7, 8 of 9 and 8 clear brains of 10 survivors respectively. Clearance of infection in other organs in survivors was similar.

All decreased drinking after infection, causing nikZ mice to ingest less than desired dose in early therapy. Despite this, they recovered sufficiently to resume pre-infection rates of drinking and thus consuming the designed drug intakes. Thus, when sickest, even less than designed dose was sufficient to promote and ultimately enable recovery.

This efficacy supports development of sustained release nikZ. Decreased intake wouldn't be a factor in humans, receiving drug via extended-release pill or continuous intravenous infusion. In prior studies (BID nikZ dosing) of murine coccidioidal meningitis, results were inferior. That suggests sustained release may provide both convenience and superior outcomes.

### BRIEF DESCRIPTION OF THE FIGURES

FIG. 1 shows a survival chart for the tested therapeutic regimens.

FIG. 2 shows colony forming unit (CFU) burdens in brain.

FIG. 3 shows water consumed as a fraction of the intended drinking, which reflects the fraction ingested of the intended dose, by day.

FIG. 4 shows water consumed as a fraction of the intended drinking, which reflects the fraction ingested of the intended dose, by day, from a previous, disseminated disease model study (U.S. patent application Ser. No. 17/719,425).

FIG. 5 shows the count of fungal colony forming units (CFU), on a log₁₀ scale, by organ (lung, liver and spleen) and by therapy group, plotted as a dose response curve for actual doses consumed, from that Ser. No. 17/719,425 previous disseminated disease study.

FIG. 6 shows the dose response curve for CFU in lung, also including a fitted sigmoid curve showing a very good fit from the lowest doses tested up through the 407 mg/kg/day dose, an extract of FIG. 5 plus the fitted curve.

FIG. 7 shows the dose response curve for CFU in liver, also including a fitted sigmoid curve showing a very good fit from the lowest doses tested up through the 407 mg/kg/day dose, an extract of FIG. 5 plus the fitted curve.

FIG. 8 shows survival by daily dose administered, by DiW in this study or BID or TID in previous studies.

### DETAILED DESCRIPTION

### Introduction

The background section notes that coccidioidal meningitis is a serious disease with poor therapy options. Nikkomycin Z (nikZ) is particularly inhibitory to the dimorphic fungi [6-10]. Against coccidioidomycosis, striking oral nikZ antifungal efficacy has been demonstrated in murine models of pulmonary, central nervous system or disseminated disease [6, 7, 11b, 12].

A concern with possible future therapy with nikZ in humans is its half-life in blood of approximately 1-2 hours, in both mice and humans [13-15]. This has resulted in the use of multiple dose/day regimens in animal experiments, shown superior to once/day administration

### Materials and Methods

Animal studies were conducted by and at the California Institute for Medical Research, San Jose, Calif. (“CIMR”).

Materials: Nikkomycin Z (nikZ) powder was provided by Valley Fever Solutions. Fluconazole (FCZ) powder was obtained from Pfizer, Inc. (New York, N.Y.) and penicillin and streptomycin obtained from Sigma Aldrich, St. Louis Mo.

Stability of nikZ in solution at ambient temperature was studied by preparing a 10 mcg/ml solution in water, sampling at 48 and 96 hours, and each time assessing bioactivity by performing a susceptibility test against Coccidioides as described below. Stability was also assessed by hplc. A refrigerated solution of nikZ used for treating the mice showed minimal degradation over a time period of months.

Isolate: Coccidioides posadasii, Silveira strain (ATCC 28-868), originally a clinical isolate, known to be virulent in animal studies [16-20], and cultured as previously described, in mycelial form [16-20, 21], was used. All handling and testing procedures of the organism were conducted per Biosafety Level 3 requirements. The use of Coccidioides in the CIMR laboratory was approved by the California Institute for Medical Research Biological Use Committee (approval no. 001-03 Yr.14), and the Institutional Animal Care and Use committee (approval no. 18-01:03).

In vitro susceptibility (MIC) and fungicidal (MFC) testing: Macrobroth dilution was performed with arthroconidia in tubes, for MIC and MFC determinations as previously described [19, 21, 24, 25]. In brief, the initial inoculum was 103 arthroconidia/mL with a two-fold dilution range, starting at 100 mcg/ml for FCZ and 20 mcg/ml for nikZ, and incubated at a temperature to approximate human body temperature (35-37° C.). The MIC value was determined at 7 days when the control growth was 4+, and the endpoint was defined as the first tube without growth (trailing endpoints were not seen with this isolate). MFC was determined by sub-culturing the isolate to agar and defined as killing ≥99% of the initial inoculum. The reading occurred after 7 days of incubation.

Animals: Female CD-1 mice, 6 weeks of age at the onset of each study, were obtained from Charles River (Wilmington, Mass.). Ten mice per treatment group were housed in groups of five in micro isolator cages. Sterilized food and acidified water were provided ad libitum to all groups prior to treatment.

Infection: Coccidioides infection was established by a technique described previously [22]. Briefly, mice were sedated by injection of dexmedetomidine hydrochloride according to body weight, and anesthetized by isoflurane inhalation. Eighty-two arthroconidia in a volume of 50 μl was inoculated intracerebrally at a point midline on the cranium, 4 to 5 mm posterior to the eyes [23]. A 27-gauge disposable needle was used to deliver the inoculum to a depth of 2 to 3 mm. Mice were injected intraperitoneally with atipamezole hydrochloride according to body weight, and fully recovered within 5 min of the procedure.

Treatment: Therapy was begun on the third day after the day of infection, and continued for 12 days. Treatment was administered by gavage (FCZ) once daily, or orally in drinking water (nikZ). It was determined that healthy uninfected mice, of this sex, age and strain, drink an average intake of 4.6 ml/mouse/day of acidified water. For the nikZ treated animals, this volume of water, containing drug, was assured to be available to them, and that no cage was without water at any time. Water bottles were changed every 3 or 4 days. Actual intake per cage was monitored on a daily basis and used to calculate the average drug intake in a group, per mouse. It has to be taken into account that the actual water intake by individual mice in a cage could vary, and almost certainly does.

The 5 study groups were FCZ 100 mg/kg/day/mouse; designed doses for the nikZ groups, 30, 100 or 300 mg/kg/day/mouse (n=10 for treatment groups); and control (no treatment) (n=9).

Study end points: Following treatment, mice were observed for another 30 days, receiving rodent chow (irradiated Teklad Global 16% Rodent Diet; Envigo, Livermore, Calif.) and drinking water ad libitum. Survival of mice was monitored throughout the studies. On day 45 post-infection all surviving mice were euthanized by CO2 asphyxiation, and brain, lungs, liver, kidneys and spleen were recovered. Mice that died prior to this (with one exception, discussed below) were not cultured, because of the uncertainty of effect on fungal growth in organs of mice discovered in a cage after time elapsed after its death.

Whole brains were homogenized in 5 ml of 0.9% sterile saline, containing penicillin and streptomycin. From the homogenate, 0.5 ml was cultured onto agar, in duplicate, and 0.5 ml used to make serial 10-fold dilutions in saline, for further quantification. Of the dilutions, 0.05 ml were cultured on Sabouraud Dextrose Agar (BD, Franklin Lakes, N.J.; SDA) in duplicate. The other organs were homogenized, and the total volume plated on SDA. The number of CFU/plate of the latter was enumerated, and four groups were noted, as follows: 0, no colonies; 1-2 colonies; 10-20 colonies; considerably >20 colonies (overgrown, too numerous to count, individual colonies not countable).

Statistical analysis: Survival differences were analyzed by the log rank test [26]. For assessment of brain sterilization and its consequences, non-survivors were assigned a worse outcome score than survivors with any residual CFU, in accordance with handling of censored data [27, 28], and then assessed by the Mann-Whitney nonparametric test [26]. For assessment of infection in other organs, which could only include data on survivors, the CFU data fell into 4 clear groups as noted above, and because of the paucity of values in some table cells, required the Fisher exact test [26]. GraphPad Prizm 9.0.1 (GraphPad Software, San Diego, Calif.) was helpful, after data entry, in directing to the appropriate tests to use.

### Results

In vitro susceptibility of isolates: In the previous U.S. Patent App No. 63/174,537 and Ser. No. 17/719,537 study [and see 11b], the MIC and MFC of the Coccidioides isolate used was determined to be 6.25 and >100 mcg/ml for FCZ, respectively, and 2.5 and 2.5 mcg/ml for nikZ. Bioactivity of nikZ, assessed as described in Methods, was unchanged in water at ambient temperature over 96 hrs.

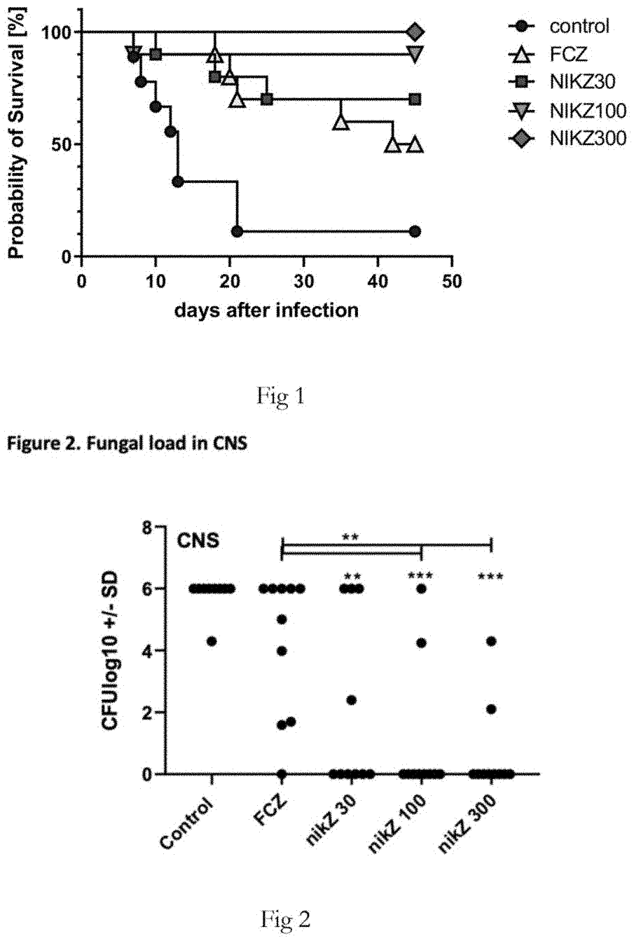

Survival: The challenge was highly lethal (FIG. 1), with 89% of untreated mice succumbing. Survival in all treated mice was significantly improved compared to controls: FCZ, nikZ 30, 100 and 300 mg/kg/day, p=0.01, 0.005, 0.001, <0.0001, respectively. The two higher nikZ dose groups, 100 and 300 mg/kg/day, were superior to FCZ, p=0.08 and 0.01, respectively. NikZ 300 mg/kg/day was superior to 30 mg/kg/day, p=0.07.

Stability of nikZ

As per the testing described in Methods, bioactivity of nikZ was unchanged in water at ambient temperature over 96 hrs (MIC and MFC as per preceding paragraph).

Sterilization, brains: The residual brain infection in survivors is shown in FIG. 2. All nonsurvivors were presumed dead from brain infection +/−infection in other organs. As FIG. 2 indicates, brains were sterilized in ≥6 mice/10 treated with any nikZ dose. Of the four treatment groups, only the three nikZ groups were significantly different than control, p≤0.01-0.001. The two higher nikZ dose groups, 100 and 300 mg/kg/day, were both superior to FCZ, p≤0.01.

Sterilization, other organs (survivors only). Dissemination from the brain to other organs was detected after inoculum was injected into the brain. Infection spread to lung, liver, spleen and kidney (Table 1A-1D). In three of the organs (lung, liver and spleen) in survivors, nikZ 100 and 300 mg/kg/day were both significantly superior to FCZ, p<0.03-0.005, and in spleen, nikZ 30 mg/kg/day was also superior to FCZ, p<0.05. Kidneys were cleared effectively in all survivors by all therapies. The paucity of survivors in the control and FCZ groups would tend to underestimate differences from nikZ groups.

Owing to several cage examinations during the day, one FCZ-treated mouse was found shortly after its death, and necropsied. Semiquantitative culture of its organs indicated CFU too numerous to count in all organs, except the spleen (no CFU) (data not included in Table 1, for survivors).

TABLES 1A-1D
Lung, Liver, Spleen
Table 1A-Lung-CFU in survivors

| CFU | Control | FCZ | nikZ 30 | nikZ 100 | nikZ 300 |
|---|---|---|---|---|---|
| 0 | 0 | 1 | 6 | 9 | 9 |
| 1-2 | 0 | 2 | 1 | 0 | 1 |
| 10-20 | 0 | 1 | 0 | 0 | 0 |
| TNTC | 1 | 1 | 0 | 0 | 0 |

Table 1B-Liver

| CFU | Control | FCZ | nikZ 30 | nikZ 100 | nikZ 300 |
|---|---|---|---|---|---|
| 0 | 0 | 1 | 6 | 9 | 10 |
| 1-2 | 0 | 1 | 1 | 0 | 0 |
| 10-20 | 0 | 2 | 0 | 0 | 0 |
| TNTC | 1 | 1 | 0 | 0 | 0 |

Table 1C-Spleen

| CFU | Control | FCZ | nikZ 30 | nikZ 100 | nikZ 300 |
|---|---|---|---|---|---|
| 0 | 0 | 2 | 7 | 9 | 10 |
| 1-2 | 0 | 2 | 0 | 0 | 0 |
| 10-20 | 0 | 1 | 0 | 0 | 0 |
| TNTC | 1 | 0 | 0 | 0 | 0 |

Table 1D-Kidney

| CFU | Control | FCZ | nikZ 30 | nikZ 100 | nikZ 300 |
|---|---|---|---|---|---|
| 0 | 0 | 5 | 7 | 9 | 10 |
| 1-2 | 0 | 0 | 0 | 0 | 0 |
| 10-20 | 1 | 0 | 0 | 0 | 0 |
| TNTC | 0 | 0 | 0 | 0 | 0 |

Overall therapeutic results Comprehensive Table 2 shows the overall survival and sterilization results, with enumeration of infection by organ. That dissemination to other organs arises from the brain is supported by the finding that for all those surviving animals for which brain was sterilized, no other organs were infected. Complete sterilization (all organs) was documented in 0, 1, 6, 8, 8 mice in the control, FCZ, and nikZ 30, 100 and 300 mg/kg/day groups, respectively.

TABLE 2
Survival, number of mice showing any infection (mostly sterilized)

| | | | Infected | | | | | | |
|---|---|---|---|---|---|---|---|---|---|
| | | | brain and | | | | | | |
| | | | infected | Infected | | | | | No |
| | | Infected | other | brain | Infected | Infected | Infected | infected | infection |
| Groups | Survivors | brain*\hat{ } | organs* | only* | lung* | liver* | spleen* | kidney* | any organ |
| control | 1/9 | 1 | 1 | 0 | 1 | 1 | 1 | 1 | 0 |
| FCZ | 5/10 | 4 | 4 | 0 | 4 | 4 | 3 | 1 | 1 |
| NIKZ | 7/10 | 1 | 1 | 0 | 1 | 1 | 0 | 0 | 6 |
| 30 | | | | | | | | | |
| NIKZ | 9/10 | 1 | 0 | 1 | 0 | 0 | 0 | 0 | 8 |
| 100 | | | | | | | | | |
| NIKZ | 10/10 | 2 | 1 | 1 | 1 | 0 | 0 | 0 | 8 |
| 300 | | | | | | | | | |

NikZ groups are assigned doses, mg/kg/day
*among survivors.
\hat{ }Survivors without brain CFU had no other organs infected. One nikZ 300 group brain-infected survivor had only 1 CFU in lung and no other organs infected.

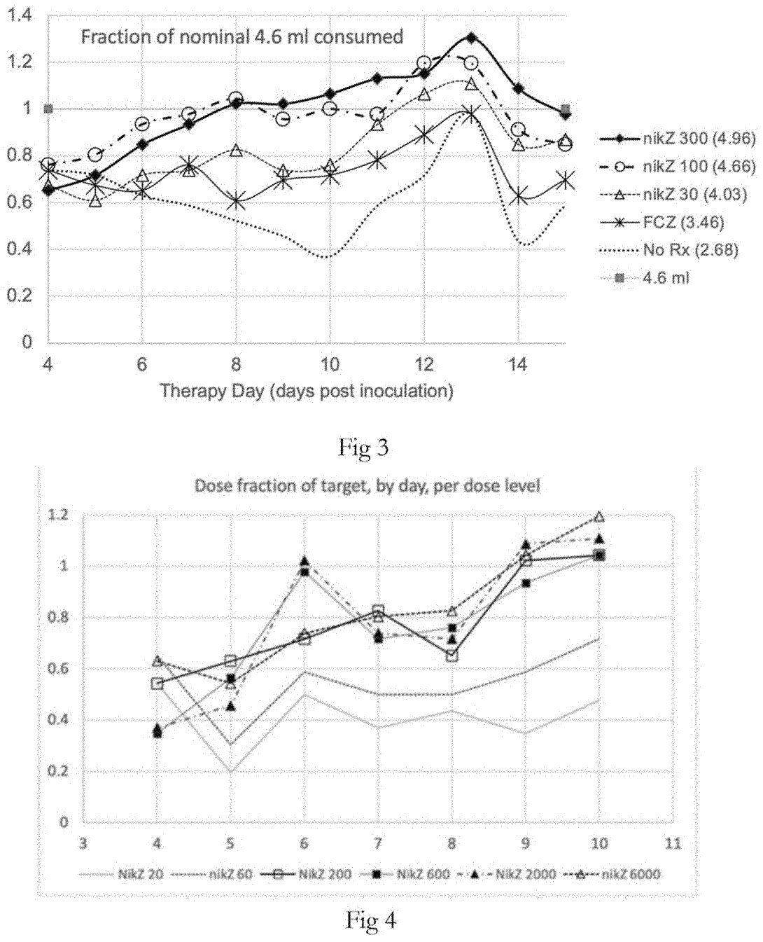

Modeling sustained release dosing Oral water intake was measured during each treatment day for each cage of 4-5 mice, to assure each day's doses, as designed for each group, were taken in full if possible, and that no mice were without water at any time. The calculated water intake/mouse was adjusted for the number of mice in a cage that died during the observation period. Previous studies, U.S. Patent App No. 63/174,537 and Ser. No. 17/719,425, indicated no aversion to nikZ in drinking water, even at much higher dosing than used in the present study, and this was corroborated in the present study (Table 3). However, assuredly related to the severity of infection and/or central nervous system effects of inflammation or an expanding brain mass, water intake in all groups was less than the 4.6 ml/d intake per uninfected mouse, for most of their course. This decreased intake includes the two ad libitum water groups, control and FCZ. The water intake data becomes a critical issue for the nikZ mice, who were getting their therapy only in their drinking water. This data (Table 3) incidentally is also, in general terms, a marker of recovering health and shows recovery over time in successfully treated groups and shows the effects of different treatments. See FIG. 3.

TABLE 3
Water intake per surviving mouse/24 hours, in each treatment group.
All numbers except DPI (days post infection) in [mL]

| DPI* | Control | FCZ | NikZ 30 | NikZ 100 | NikZ 300 |
|---|---|---|---|---|---|
| 4 | 3.4 | 3.4 | 3.1 | 3.5 | 3.0 |
| 5 | 3.3 | 3.1 | 2.8 | 3.7 | 3.3 |
| 6 | 2.9 | 3.0 | 3.3 | 4.3 | 3.9 |
| 7 | 2.7 | 3.5 | 3.4 | 4.5 | 4.3 |
| 8 | 2.4 | 2.8 | 3.8 | 4.8 | 4.7 |
| 9 | 2.1 | 3.2 | 3.4 | 4.4 | 4.7 |
| 10 | 1.7 | 3.3 | 3.5 | 4.6 | 4.9 |
| 11 | 2.7 | 3.6 | 4.3 | 4.5 | 5.2 |
| 12 | 3.3 | 4.1 | 4.9 | 5.5 | 5.3 |
| 13 | 4.5 | 4.5 | 5.1 | 5.5 | 6.0 |
| 14 | 2.0 | 2.9 | 3.9 | 4.2 | 5.0 |
| 15 | 2.7 | 3.2 | 4.0 | 3.9 | 4.5 |

DPI = days post infection, from start of therapy.
NikZ groups are assigned doses, mg/kg/day

The data of Table 3 are graphed in FIG. 3, as a fraction of the nominal intake of 4.6 ml/mouse/day. All infected mice consume less than the expected amount of water (medicated for the nikZ groups). In general, these improve day over day during therapy for all groups, which could be reasonably correlated with improved activity and health in the treated animals. This is not a tight correlation but does show a general trend. Even the FCZ treated group improve in a similar manner. FIG. 4 illustrates the pattern in the previous study, high inoculum disseminated coccidioidomycosis, with a very high inoculum challenge, and treated for only 5 days. U.S. Pat App. No. 63/174,537 and Ser. No. 17/719,425.

As a result of the reduced intake of water, the doses received for the nikZ-dosed mice only approximated the targeted doses. Table 4 shows the actual doses in these groups.

TABLE 4
Comparison of desired and calculated actual drug intake
per surviving mouse/24 hours in each treatment group.
All numbers except DPI in [mg/kg].

| DPI* | NikZ 30 | NikZ 100 | NikZ 300 |
|---|---|---|---|
| 4 | 20.35 | 76.96 | 194.35 |
| 5 | 18.07 | 81.09 | 212.61 |
| 6 | 21.78 | 94.35 | 252.39 |
| 7 | 22.17 | 98.26 | 277.17 |
| 8 | 24.72 | 105.00 | 306.52 |
| 9 | 21.91 | 97.01 | 303.26 |
| 10 | 24.93 | 100.00 | 320.87 |
| 11 | 27.65 | 98.32 | 337.17 |
| 12 | 31.55 | 118.26 | 343.70 |
| 13 | 33.33 | 119.35 | 388.04 |
| 14 | 24.70 | 90.60 | 324.13 |
| 15 | 25.42 | 83.91 | 294.78 |
| Mean | 24.72 | 96.93 | 296.25 |

DPI = days post infection, from start of therapy.
NikZ groups (column headings) are given as desired doses, mg/kg/day. Numbers in table are actual doses, average per mouse.

The average ingested dose closely approximated the designated dose overall, particularly in the two highest nikZ groups. What is also apparent is that their initial drug intake at the start of therapy, when they were sickest, was considerably below that desired, concluding that even much lower doses than programmed were sufficient to start these animals on the road to recovery. For example, on the initial treatment day, only approximately two thirds of the target dose was ingested. After they gained some therapeutic momentum against their disease, they then approached typical pre-infection drinking rates, and with that approximated designed drug intake, to continue their therapy. This appeared to stabilize by therapy day 3 (Table 3, 4, FIG. 3).

### Clean Dose Response to DiW Dosing (Disseminated Disease Model)

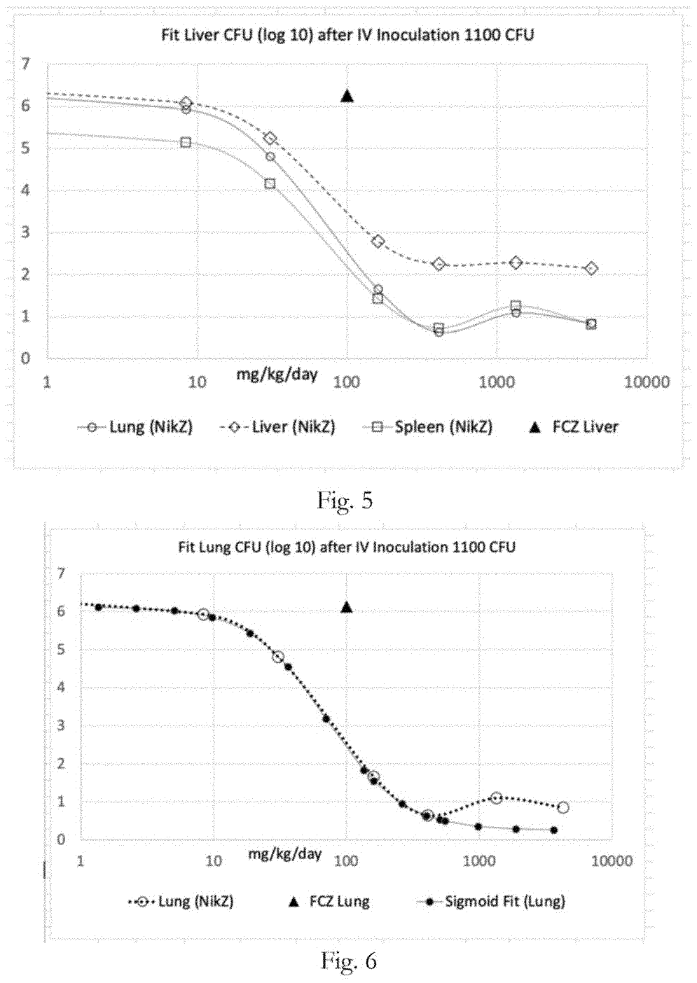

In a previous study of disseminated disease, U.S. Patent App No. 63/174,537, the reduction of CFU in lung, liver and spleen shows a clean dose response to nikZ DiW dosing. Referring to FIG. 5, the ordinate is a count of CFU by log₁₀. The abscissa (dose, mg/kg/day) is logarithmic, so a zero value does not plot well, Setting the “zero” point at 0.001 gave the curves shown. For lung and liver, the untreated organs were considered to have a count of 10\hat{ }7 (7 log₁₀) and spleen 10\hat{ }6 CFU.

The dose response curve for each organ fits a sigmoid curve very well for all but the highest two doses, with generally similar parameters for each organ. For spleen and lung, fungal growth was highly suppressed, to counts below 10 (1 log₁₀) CFU, with an asymptote close to zero. For liver, the asymptote was about 100 (2 log₁₀). Perhaps extending therapy for more than 5 days would further reduce this count, as nikZ is more effective in other organs.

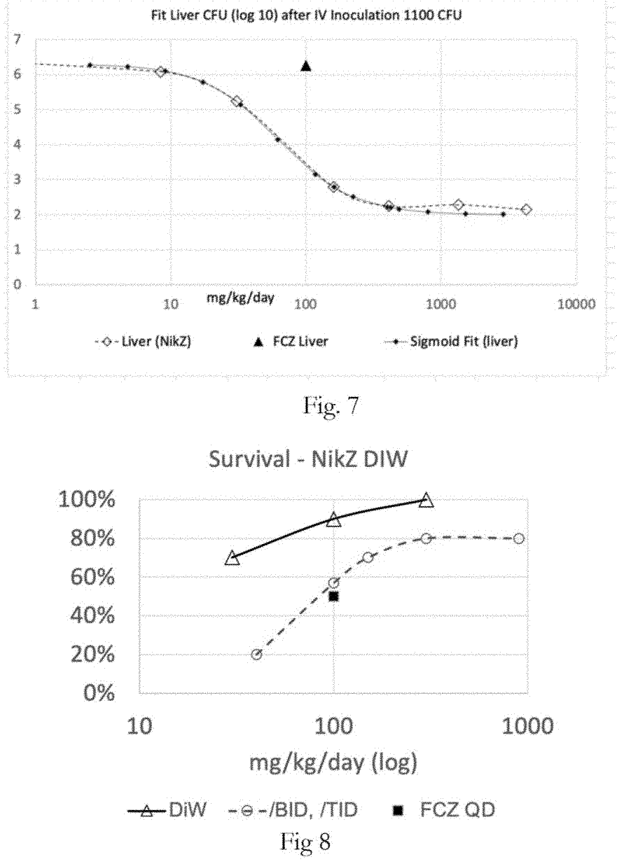

Referring to FIG. 6, plotting just the observed lung CFU counts and fitting a sigmoid curve shows the extrapolated terminal asymptotic pattern, and highlights the deviation in the two highest doses, which were effective but not fitting the dose response curve. Referring to FIG. 7, similarly plotting just the liver and fitting a sigmoid curve, the highest two doses do not monotonically fit the dose response, although here the highest dose is close to the expected terminal value.

### Discussion

NikZ protected against a highly lethal coccidioidal challenge to the CNS [7], by the oral route. The study showed the superiority of nikZ, whether compared on a mg/kg or molar basis, in both survival and in reducing or eliminating the CNS infection, to FCZ, the most commonly used drug in this disease [2], for this most severe manifestation of coccidioidal infection. Other drugs, and other approaches to therapy, in human coccidioidal meningitis, are reviewed elsewhere [2-4].

Nik Z was also effective in reducing or eliminating the disseminated infection, and more effective than FCZ. This was not unexpected, as a prior study of disseminated coccidioidomycosis, after intravenous challenge has already indicated efficacy of nikZ treatment in those affected organs. U.S. Patent App. No. 63/174,537 and Ser. No. 17/719,425 [and see 11b]. Efficacy of nikZ against pulmonary coccidioidal challenge has also previously been shown [6].

In the previous study of nikZ against disseminated coccidioidomycosis, U.S. Patent App No. 63/174,537 and Ser. No. 17/719,425, dosing only in drinking water matched the efficacy of parenteral dosing of nikZ twice daily, and both routes were superior in efficacy to FCZ [11b]. Consistent with this drug delivery efficacy is the observation that divided oral doses produced a superior result to once daily dosing in the pulmonary coccidioidomycosis study [6].

In a previous study of brain intraparenchymal coccidioidal infection [7], employing an almost identical inoculum challenge (with the same fungal strain) and duration of observation, and despite treating almost twice as long as in this study and initiating therapy earlier than in this study, produced 60% survival with 100 mg/kg/day nikZ divided into oral twice daily administration. Table 5. A second similar study [12], with a similar inoculum, but treatment now almost 5 times the length of this study, and a similar post-treatment observation period to this study, again produced 60% survival with 100 mg/kg/day nikZ divided into oral twice daily administration. A third similar previous study for an intermediate duration produced slightly lower 50% survival with the same dose. Those two results are inferior to the 90% survival with the current study 100 mg/kg/day via semi-continuous drinking. That suggests, in addition to the possible convenience of sustained release in future, that sustained release may be a superior therapeutic modality.

TABLE 5
100 mg/kg, various dosing methods

| Dose | Treatment Days | Survival |
|---|---|---|
| BID | 21 | 60% |
| BID | 56 | 60% |
| BID | 44 | 50% |
| DiW | 12 | 90% |

FIG. 8 shows this in a broader context. Dosing by DiW at 30 mg/kg/day gave 70% survival. Dosing by BID at 40 mg/kg/day gave 20% survival. Dosing at BID 600 or TID 900 mg/kg/day gave 80% survival. This illustrates the higher impact of divided dosing. Even finer and more precise dosing in an extended-release format should give results at least as good as DiW and very likely better (better survival at lower doses).

This efficacy in this study supports the development of a sustained release form of the drug in humans. This would counter the short half-life of the drug in blood [13-15]. Here, sustained release was mimicked by providing high frequency dosing in drinking water. The efficacy of this semi-continuous dosing supports the earlier study observation showing even sick mice consume enough drug in water to acquire at least useful amounts of nikZ sufficient to result in a clinical response [11b]. This study also demonstrates the vicissitudes of such dosing regimens in experiments, where it is difficult to steadily achieve the desired dosing levels, when dependent on the consistent drinking by the animal subjects, and their degree of illness can affect their intake. This design complication would not be expected to be a dominant factor in therapy of humans, receiving the drug via an extended-release pill or by continuous intravenous infusion, where intake would be more controlled. A future extended-release form for man could also maintain drug delivery even during sleep periods, further improving blood level consistency above the results described here, since mice ingest less water during their sleep periods. The stability of nikZ in water also suggests the success of continuous intravenous infusion administration, should that route be developed for clinical use.

For reference, after oral doses of 300 mg/kg or 100 mg/kg to ICR mice, the Cₘₐₓ in plasma were reported to be 8 and 4.2 mcg/ml, respectively, the Tmax 1 and 2 hrs., and the t_{1/2} 2.3 and 2.2 hrs. [13]. In brain, the Tmax for both doses was 2 hrs., and Cₘₐₓ 0.26 and 0.20 mcg/gm, and t_{1/2} 4.4 and 6.2 hrs., respectively [13]. With the latter information, and the susceptibility data, the efficacy noted in this study with sustained release was likely contributed to by tissue accumulation over time+constant drug organ perfusion+any contribution by host immune defenses. This hypothesis requires further pharmacologic study, after long duration dosing. Regardless of the specifics of blood levels over time, the observed therapeutic results presented here are striking. Some pharmacologic data on sustained release of nikZ in rodents (rats) is available elsewhere, both from subcutaneous depot forms (single dose study) and from continuous intravenous infusion (4 days) [29].

Further clinical trials are warranted for nikZ, for an infection in humans with high morbidity and mortality [2], with an agent with oral bioavailability, limited toxicity in multiple animal studies [6-9], and the potential convenience of an extended-release form.

### Dosing Method—Analysis

An important question that is not readily answerable in full is a detailed pharmacokinetic (PK) profile of the provided dose. The dosing method is demonstrably effective clinically, certainly among and perhaps the most effective therapies ever reported against disseminated coccidioidomycosis. Cross referenced priority application Provisional Application No. 63/061,789 (the “789 application”) discusses a target sustained release formulation. It will be easier to study precise pharmacokinetics with that formulation. That '789 application discusses in some detail modeling how a high frequency (Q≤2 hour) repeat dosing schedule for a highly divided daily dose would reduce variation of blood nikZ levels around the steady state level for a given dosing regimen. From PK measurements in mice, dogs and humans, a single oral dose reaches Cₘₐₓ in 1-2 hours (depends on the size of the dose) and decays to about 5% of Cₘₐₓ by 8 hours. See Table 6. Repeat dosing at Q 8 hours will thus be additive, but only slightly. At 4 hours (3 hours post Cₘₐₓ peak in a mouse), the blood level is 37% of Cₘₐₓ and at 2 hours the blood level is 62% of Cₘₐₓ, so for any dosing at Q<4 hours, the additive effective is significant, and Cₘₐₓ is significantly higher than with BID dosing (Cₘᵢₙ 1% of Cₘₐₓ)

TABLE 6
Mouse plasma nikZ levels, 50 mg/kg single
dose PK (TID, Q 8 hr repeat, 7^{th} day)

| time | ng/ml | % |
|---|---|---|
| 0.5 | 2920 | 77.9 |
| 1 | 3750 | 100 |
| 2 | 2320 | 61.9 |
| 4 | 1400 | 37.3 |
| 8 | 180 | 4.8 |
| 12 | 35 | 0.9 |
| 24 | 7.5 | 0.2 |

For the DiW dosing, a first mouse takes sip #1 of size 1 at time 1, then sip 2 of size 2 at time 2, etc. Each mouse in the cage has its own pattern. This quickly becomes extremely complex, so it becomes impractical to measure except at the population level. Fortunately, for a range of time from 0.5 to about 1.5 hours after a sip, the blood drug level contribution from that sip is within 75% of maximal. A next sip within 4 hours will be noticeably additive, and more additive if taken within a shorter time. Sips of varying sizes will contribute additively, with best additive effect when taken within about an hour of a previous sip.

This simplifies greatly by looking at the clinical results. Knowing that a cage of mice will drink an expected amount of water over 24 hours and dividing that among mice in the cage, and assuming each mouse consumes a generally similar quantity of water makes it easy to measure and calculate the average water consumed per cage and thus per mouse per measurement period.

The size of each episodic drink can exceed efficient oral uptake.

TABLE 7
Mouse plasma PK, dose levels 50, 100, 300 mpk single oral dose

| dose | T 1/2 | Tmax | Cmax | SE | AUC | SE |
|---|---|---|---|---|---|---|
| 50 | 2.61 | 1 | 3,750 | 386 | 13,000 | 785 |
| 100 | 2.15 | 1 | 4,620 | 385 | 20,600 | 1,590 |
| 300 | 2.26 | 2 | 7,950 | 372 | 38,200 | 1,440 |

Noting from Table 7 that the C_{max does not scale proportionally even from} 50 to 100 mg/kg, (62% of the linear-scaled Cₘₐₓ 9,240 ng/ml expected at double the dose) and worse at 300 (35%), it appears there is a reduction in oral uptake efficiency at least above 50 mg/kg and perhaps from some lower point. A similar tapering in uptake efficiency is observed in humans at about 5.5 mg/kg. Since allometric scaling from mouse human is frequently calculated in the range of 8 to 10 (50 mg/kg in a mouse correlates to 5-6.25 mg/kg in a human), this is a good correlation. The breakpoint in humans was calculated from considerable data. Nix 2009 [33].

Putting all of this together, in the high inoculum challenge of U.S. Patent App. No. 63/174,537 and Ser. No. 17/719,425, doses up to 407 mg/kg/day fit very well on a dose response curve, with less-than-projected additional clinical benefit from the next higher dose of 1334 mg/kg/day (3.27× higher dose, but less clinical response).

For this CNS study, the maximum dose used was 300 mg/kg/day, well within the region of linear dosing by nikZ DiW shown in that earlier '425 study. Referring to FIGS. 5-7, even 30 mg/kg/day showed −1.7, −1.8 and −3.2 log₁₀ CFU reduction in liver, spleen and lung respectively.

Not relevant for this CNS study, but perhaps for considering highest doses, assuming arguendo that 50 mg/kg/sip is an efficient dose, 400 mg/kg divided by 50 would be 8 such sips. Over a 16-hour waking period of drinking, this would be an average of such a sip every two hours. Considered by volume, dividing 4.6 ml/day into 8 subunits averaged to 0.575 ml.

This does not mean this is an actual pattern, and such regularity is unlikely, but such a pattern would fall within the general constraints of dosing Q≤˜2 hours at a maximum of 50 mg/kg dose. Treating this as a population average is at least consistent with the clinical benefit dose response curve. Drinking could be faster, or smaller, and is almost certainly not this regular. Doses with sufficient spread in quantity and time will add cleanly if small or infrequent enough but at some point of increasing size or frequency may hit saturation limits.

If drinking is on average about 8 sips per day, a dose 3.27× higher would be significantly in the non-linear oral uptake region. This would imply a less efficient oral uptake, and corresponding non-linear improvement in fungal burden reduction at this higher dose. Then there are issues of dose tolerability, animal health and much more that might disincentivize drinking the daily dose of 1334 mg/kg. The measured intake suggests the intake quantity is likely similar at all doses above 160 mg/kg/day, so some sort of uptake efficacy limit or simply pharmacodynamics such as exceeding a therapeutic dose for no additional clinical benefit may contribute to the non-linearity at doses higher than 407 mg/kg in this study.

Fortunately for this model, this strain, and this drug, dosing at 407 mg/kg/day is quite effective. Extrapolation suggests that the linear limit could be somewhat higher, perhaps in the 500-600 mg/kg/day range. Regardless of the PK details, the dosing is very effective. A future sustained release formulation will be more amenable for detailed study.

Considering possible alternative dosing schemes, mice will drink water from a controlled source, but other animals will not. This is particularly complicated in communities of animals sharing resources. If a pet dog is to be treated but shares a water supply with other animals that do not need therapy, a shared water bowl may not be ideal. If dosing a dog by hand, dosing more often than twice a day (Q 12 hours) becomes challenging for a caretaker, particularly when dosing for months. This is only made worse if the therapy is given Q<12 hours. Nevertheless, some caregivers are willing to dose at Q 4 hours. To address sleep cycles, the mid-sleep cycle dose can be given in advance at bedtime or in the morning to “catch up” to the dosing scheme. For a motivated caregiver this can be even more frequent, such as Q 2 hours, with the daily dose divided 24/2 or 12 dose units. A convenient pattern here is to target dosing to even hours, 6, 8, 10 am until bedtime. For the doses during sleep period, some can be advanced to the last dose of the day, which could be 8 or 10 pm or even an arbitrary hour. For the wake up first dose of the next day, any pending catch up dose units can be given with the 6 or 8 am dose, or again at an arbitrary hour. Dividing the sleep dose units evenly between pre- and post-sleep is a fine approach. Favoring the pre-sleep dose time slightly and administering up to 75% of the sleep period dosing before sleep, even 100% if desired, will still be useful.

A human can follow such a Q 2 hour pattern as well. A human can divide even more finely. In a simple variation on DiW, a period dose, such as 8 hours (33% of the day) can be poured into a simple drinking container. The patient sips this periodically, keeping rough track of the pace of drinking. This becomes even easier by using a graduated drinking container. This does not have to be the patient's only drinking water. Taking the recommended “quart a day” and allocating about 25% to drug dosing, this could easily be 240 ml for drug. The target dose is divided into a 1 hour dose in 10 ml, and the patient is asked to drink that episodically, paying particular attention to catching up at convenient times, such as every 2-4 hours. This can be even more convenient if the dose vessel is calibrated, such as a graduated cylinder or typical kitchen measuring vessels. See US Pat App. No. 63/061,789.

To deal with sleep period, approximately 4 hours of dosing can be consumed shortly before bedtime, and in the morning catch up to the clock schedule. To minimize saturating oral uptake efficiency, these larger pre- and post-sleep doses can be spread out a bit, such as over 30-60 minutes.

A planned extended-release oral dosage form will be easier on patients, but the sipping method is feasible. This could be useful, for example, in a situation where swallowing an oral dose is difficult.

Similarly, administration by IV can be conveniently continuous, quite common in a hospital setting and available for outpatients (PortaCath, etc.), but episodic bolus dosing can still give and maintain useful blood levels of nikZ.

### ACKNOWLEDGEMENTS

These studies were funded in part by Valley Fever Solutions and a grant from Valley Fever Americas Foundation of Bakersfield, Calif. NikZ used in the study was supported in part by an earlier NIH grant, U01-AI-112406, 2014-2018.

### REFERENCES

1. Chiller, T. M.; Galgiani, J. N.; Stevens, D. A. Coccidioidomycosis. Infect. Dis. Clin. N. Am. 2003, 17, 41-57.
2. Galgiani, J. N., Ampel, N., Blair, J., Catanzaro, A., Geertsma, F., Hoover, S. E., Johnson, R., Kusne, S., Lisse, J., MacDonald, J., Meyerson, S., Raksin, P. B., Siever, J., Stevens, D. A., Sunenshine, R., Theodore, N. Coccidioidomycosis. Practice Guideline, Infectious Disease Society of America. Clin. Infect. Dis. 63:717-722, 2016 (Executive Summary) and 63:e112-146, 2016 (complete).
3. Dewsnup, D. H.; Galgiani, J. N.; Graybill, J. R.; Diaz, M.; Rendon, A.; Cloud, G. A.; Stevens, D. A. Is it ever safe to stop azole therapy for Coccidioides immitis meningitis? Ann. Intern. Med. 1996, 124, 305-310.
4. Stevens, D. A.; Shatsky, S. A. Intrathecal amphotericin in the management of coccidioidal meningitis. Semin. Respir. Infect. 2001, 16, 263-269.
5. Fiedler, H.-P., Schuz, T., Decker, H. An overview of nikkomycins: history, biochemistry, and applications. Chapter 24 in Cutaneous Antifungal Agents, J. W. Rippon and R. A. Fromtling, eds., Marcel Dekker, Inc., pub. 1993
6. Shubitz, L. F., Trinh, H. T., Perrill, R. H., Thompson, C. M., Hanan, N. J., Galgiani, J. N., Nix, D. E. Modeling nikkomycin Z dosing and pharmacology in murine pulmonary coccidioidomycosis preparatory to phase 2 clinical trials. J. Infect. Dis. 209:1949-1954, 2014. doi: 10.1093/infdis/jiu029. PMID: 24421256; PMCID: PMC4038145.
7. Hector, R. F., Zimmer, B. L., Pappagianis, D. Evaluation of nikkomycins X and Z in murine models of coccidioidomycosis, histoplasmosis, and blastomycosis. Antimicrob. Agents Chemother. 34:587-593, 1990.
8. Clemons, K. V., Stevens, D. A. Efficacy of nikkomycin Z against experimental pulmonary blastomycosis. Antimicrob. Agents Chemother. 41:2026-2028, 1997.
9. Larwood, D. J. Nikkomycin Z-Ready to meet the promise? J. Fungi (Basel) 6:261, 2020.
10. Poester, V. R., Munhoz, L. S., Larwood, D., Martinez, M., Stevens, D. A., Xavier, M. O. Nikkomycin Z, and its interaction with itraconazole, against Sporothrix spp. Med. Mycol., 2020.
11. Sass, G., Larwood, D., Martinez, M., Chatterjee, P., Xavier, M. O., Stevens, D. A. Nikkomycin Z against disseminated coccidioidomycosis in a murine model of sustained release dosing. 64th Ann. Coccidioidomycosis Study Group Meeting, 2020, Abstract no. 5.
11b. Sass G, Larwood D J, Martinez M, Chatterjee P, Xavier M O, Stevens D A. Nikkomycin Z against Disseminated Coccidioidomycosis in a Murine Model of Sustained-Release Dosing. Antimicrob Agents Chemother. 2021 Sep. 17; 65(10):e0028521.
12. Hector, R. F., Zimmer, B. L., Pappagianis, D. Nikkomycins, in Recent Progress in Antifungal Chemotherapy; H. Yamaguchi, G. S. Kobayashi, H. Takahashi, eds., Marcell Dekker, pub., New York, 1992 (p. 341-353)
13. Catz, P., 2020, private communication.
14. Swezey, R. R., Hector, R. F., Khandwala, A., Sinha, S., Carlson, T., Charney, M. R. Pharmacokinetics and safety of nikkomycin Z after oral administration of single doses to normal volunteers. Interscience Conference on Antimicrobial Agents and Chemotherapy, 1997, Abstract F-87
15. Nix, D. E., Hoover, S. E., Thompson, C., Hanan, N. J., Chavez, S. E., Galgiani, J. N. Pharmacokinetics and safety of nikkomycin Z following multiple rising doses in healthy subjects. Interscience Conference on Antimicrobial Agents and Chemotherapy, 2010, Abstract A1-031
16. Clemons, K. V., Hanson, L., Perlman, A., Stevens, D. A. Efficacy of SCH39304 and fluconazole in a murine model of disseminated coccidioidomycosis. Antimicrob. Agents Chemother. 34:928-930, 1990.
17. Lutz, J., Clemons, K. V., Aristizabal, B., Stevens, D. A. Activity of the triazole SCH 56592 against disseminated murine coccidioidomycosis. Antimicrob. Agents Chemother. 41:1558-1561, 1997.
18. Clemons, K. V., Stevens, D. A. Efficacies of two novel azole derivatives each containing a morpholine ring, UR-9746 and UR-9751, against systemic murine coccidioidomycosis. Antimicrob. Agents Chemother. 41:200-203, 1997.
19. Clemons, K. V., Homola, M. E., Stevens, D. A. Activities of the triazole SCH 51048 against Coccidioides immitis in vitro and in vivo. Antimicrob. Agents Chemother. 39:1169-1172, 1995.
20. Clemons, K. V., Stevens, D. A. Utility of the triazole D0870 in the treatment of experimental systemic coccidioidomycosis. J. Med. Vet. Mycol. 32:323-326, 1994.
21. Stevens, D. A., Aristizabal, B. H. In vitro antifungal activity of novel azole derivatives with a morpholine ring, UR-9746 and UR-9751, and comparison with fluconazole. Diag. Micro. Infect. Dis. 29:103-106, 1997.
22. Chiller T M, Sobel R A, Luque J C, Clemons K V, Stevens D A. Efficacy of amphotericin B or itraconazole in a murine model of central nervous system Aspergillus infection. Antimicrob Agents Chemother. 2003 February; 47(2):813-5.
23. Chiller, T. M., J. Capilla Luque, R. A. Sobel, K. Farrokhshad, K. V. Clemons, and D. A. Stevens. 2002. Development of a murine model of cerebral aspergillosis. J. Infect. Dis. 186:574-577.
24. Clinical and Laboratory Standards Institute (CLSI). Reference Method for Broth Dilution Antifungal Susceptibility Testing of Filamentous Fungi, 3rd Edition, M38, 3rd ed., pub., Annapolis Junction, M D, 2017.
25. Denning, D. W, Hanson, L. H., Perlman, A. M., Stevens, D. A. In vitro susceptibility and synergy studies of Aspergillus species to conventional and new agents. Diag. Micro. Infect. Dis. 15:21-34, 1992.
26. Brown, B. W., Hollander, M. Statistics. John Wiley and Sons, New York, pub. 1977
27. Lachin J. 1999. Worst-rank score analysis with informatively missing observations in clinical trials. Control Clin Trials 20:408-22.
28. Shih W. 2002. Problems in dealing with missing data and informative censoring in clinical trials. Curr Control Trials Cardiovasc Med 3:4.
29. Hector, R. F., Sabouni, A. U.S. Pat. No. 5,789,387, Methods and Compositions for

Treating Fungal Infections in Mammals, 1998
30. Stevens D A, Martinez M, Sass G, Pappagianis D, Doherty B, Kutsche H, McGuire M. Comparative Study of Newer and Established Methods of Diagnosing Coccidioidal Meningitis. J Fungi (Basel). 2020 Aug. 4; 6(3):125.
31. Sass, G., Larwood, D., Martinez, M., Shrestha, P., Stevens, D. A. Efficacy of Nikkomycin Z in Murine Central Nervous System Coccidioidomycosis, Modeling Sustained Release Dosing, 65th Ann. Coccidioidomycosis Study Group Meeting, Mar. 1, 2021, Abstract no. 7.
32. Sass G, Larwood D J, Martinez M, Shrestha P, Stevens D A. Efficacy of nikkomycin Z in murine CNS coccidioidomycosis: modelling sustained-release dosing. J Antimicrob Chemother. 2021 Sep. 15; 76(10):2629-2635.
33. Nix D E, Swezey R R, Hector R, Galgiani J N. Pharmacokinetics of nikkomycin Z after single rising oral doses. Antimicrob Agents Chemother. 2009; 53(6):2517-21.

What is claimed is:

1. A method of dividing a daily dose of nikkomycin-Z so the drug reaches the blood stream of a patient spread out over time into increments on average one per less than 8 hours.

2. The method of claim 1 wherein nikkomycin Z is dissolved into drinking water as a drug in water to be consumed in portions during the course of 24 hours so as to spread out the intake of drug into the body and the uptake of drug into the blood stretm.

3. The method of claim 2 wherein said drug in water is provided to a mouse as the sole source of drinking water.

4. The method of claim 3 wherein said drinking water is provided ad labium, readily available for most of the day in quantities more than needed to satisfy the animal, in other words in sufficient excess that the animal can consume as much as desired.

5. The method of claim 1 wherein the dose of nikkomycin Z is provided to an animal infected with a fungal disease.

6. The method of claim 5 wherein the fungal disease is coccidioidomycosis.

7. The method of claim 6 wherein the disease is induced by intracranial inoculation with Coccidioides.

8. The method of claim 1 wherein nikkomycin Z is dissolved into drinking water as a drug in water to be consumed in portions during the course of 24 hours so as to spread out the intake of drug into the body and the uptake of drug into the blood stream of a mammal with coccidioidomycosis.

9. The method of claim 1 wherein the coccidioidomycosis infects the brain of the mammal.

10. A method of providing a continuous source of nikkomycin-Z for consumption by a mammal,
the method comprising dissolving nikkomycin-Z in water at a concentration intended to make available a chosen level of drug per day if the mammal consumes an intended amount of water,
infecting an animal intracranially with a fatal inoculation of Coccidioides, and
treating said infected animal by providing said dissolved nikkomycin-Z in water continuously for multiple sequential days.

11. The method of claim 10 further comprising treating the infected animal with nikkomycin-Z in water for more than 3 days.

12. The method of claim 10 further comprising treating the infected animal with nikkomycin-Z in water for more than 10 days.

13. A composition of matter wherein nikkomycin-Z is dissolved in water for administration to a patient.

14. The composition of claim 13 wherein the patient is infected with a fungal disease.

15. The composition of claim 13 wherein the administration is intended to treat a fungal disease in a patient.

16. The composition of claim 13 where the fungal disease is coccidioidomycosis.
